(19) United States
(12) Patent Application Publication
(10) Pub. No.: US 2005/0032875 A1
(43) Pub. Date: Feb. 10, 2005

(54) Process for the preparation of indole derivatives

(76) Inventors: Heinz Wolleb, Fehren (CH); Annemarie Wolleb, Fehren (CH); Paul Adriaan Van Der Schaaf, Allschwil (CH); Roman Kolly, Allschwil (CH); Nicole End, Oberwil (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/487,269

(22) PCT Filed: Aug. 13, 2002
(86) PCT No.: PCT/EP02/09046

(30) Foreign Application Priority Data
Aug. 22, 2001 (EP) 01810817.5

(51) Int. Cl. C07D 209/12; A61K 31/405

(52) U.S. Cl. 514/419; 548/494

(57) ABSTRACT

A process for the preparation of compounds of formula (I) wherein R₁ is unsubstituted or substituted C₁-C₈alkyl, R₂, R₃, R₄ and R₅ are each independently of the others hydrogen, unsubstituted or substituted C₁-C₈alkyl, C₁-C₈ alkoxy, phenoxy or benzyloxy, or halogen, Y₁ and Y₂ are each independently of the other hydrogen or a protecting group, or Y₁ and Y₂ together form a protecting bridge, and X₁ is hydrogen, an organic radical or a cation, in which process a compound of formula (II) wherein R₁ R₂, R₃, R₄ and R₅ are as defined above and Z₁ is a leaving group, is reacted, in the presence of a catalytically effective amount of a palladium catalyst, with a compound of formula (III) wherein R₆ is hydrogen, bromine, chlorine, iodine, —OSO₂CF₃, —COCI, —B(OH)₂ or a mono- or di-ester derived from —B(OH)₂, Y₃ and Y₄ are each a protecting group, or Y₃ and Y₄ together form a protecting bridge, and X₁ is as defined above, to form a compound of formula (IV) and if desired the radicals Y₃ and Y₄ are converted into the radicals Y₁ and Y₂ where Y₁ and Y₂ are hydrogen.

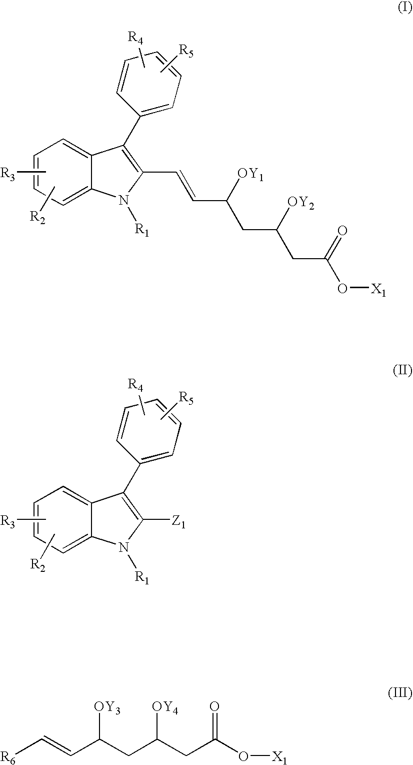

The present invention relates to a process for the preparation of indole derivatives and to novel intermediates.

Indole derivatives of the following formula (1) are known as pharmaceutical active ingredients (e.g. from U.S. Pat. No. 4,739,073) or are important precursors in the preparation thereof. An important indole derivative is fluvastatin, an HMG-CoA reductase inhibitor, that is to say an inhibitor of the biosynthesis of cholesterol, which is used in the treatment of hyperlipoproteinaemia and arteriosclerosis.

Known processes for the preparation of indole compounds of formula (1) do not in all cases meet the requirements in terms of the yield and economic viability of the processes.

The problem underlying the present Application is accordingly to provide a new process for the preparation of indole compounds of formula (1), by means of which those compounds can be obtained in as high a yield as possible combined with good economic viability.

The subject matter of the present invention is accordingly a process for the preparation of compounds of formula 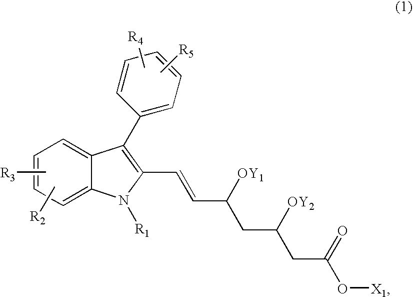
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈-alkoxy, phenoxy or benzyloxy, or halogen,
Y₁ and Y₂ are each independently of the other hydrogen or a protecting group, or Y₁ and Y₂ together form a protecting bridge, and
X₁ is hydrogen, an organic radical or a cation,
   in which process a compound of formula 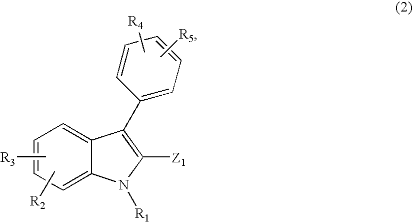
   wherein R₁, R₂, R₃, R₄ and R₅ are as defined above, and
Z₁ is a leaving group,
   is reacted, in the presence of a catalytically effective amount of a palladium catalyst, with a compound of formula 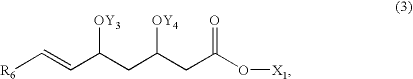
   wherein R₆ is hydrogen, bromine, chlorine, iodine, —OSO₂CF₃, —COCl, —B(OH)₂ or a mono- or di-ester derived from —B(OH)₂,
Y₃ and Y₄ are each a protecting group, or Y₃ and Y₄ together form a protecting bridge, and
X₁ is as defined above,
   to form a compound of formula 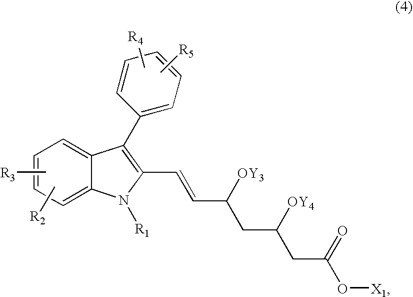
   and if desired the radicals Y₃ and Y₄ are converted into the radicals Y₁ and Y₂ where Y₁ and Y₂ are hydrogen.

As C₁-C₈alkyl radicals there come into consideration for R₁, for example, methyl, ethyl, n- or iso-propyl, n-, iso-, sec- or tert-butyl, or straight-chain or branched pentyl, hexyl, heptyl or octyl. C₁-C₄Alkyl radicals are preferred. R₁ is preferably propyl, especially isopropyl.

As C₁-C₈alkyl radicals there come into consideration for R₂, R₃, R₄ and R₅, for example, methyl, ethyl, n- or iso-propyl, n-, iso-, sec- or tert-butyl, or straight-chain or branched pentyl, hexyl, heptyl or octyl. The mentioned alkyl radicals may be unsubstituted or substituted, for example by halogen, such as fluorine. Preference is given to corresponding C₁-C₄alkyl radicals.

As C₁-C₈alkoxy radicals there come into consideration for R₂, R₃, R₄ and R₅ especially C₁-C₄-alkoxy radicals, for example methoxy or ethoxy.

As halogen there comes into consideration for R₂, R₃, R₄ and R₅, for example, fluorine or chlorine, especially fluorine.

R₂, R₃ and R₅ are preferably hydrogen. R₄ is preferably fluorine, especially fluorine bonded in the 4-position.

As protecting groups for Y₁, Y₂, Y₃ and Y₄ there may be used the groups customary for that purpose. The usual protecting groups are indicated, for example, in Protective Groups in Organic Synthesis, Th. W. Greene and P. G. M. Wuts, John Wiley & Sons, Second Edition, 1991 (especially pages 118 to 142).

Preferred as protecting groups Y₁, Y₂, Y₃ and Y₄ are C₁-C₄alkylcarbonyl or silyl radicals; there also come into consideration protecting bridges wherein Y₁ and Y₂ together or Y₃ and Y₄ together form an unsubstituted or substituted alkylene or silyl radical. Examples of C₁-C₄-alkylcarbonyl radicals that may be mentioned include methyl- and ethyl-carbonyl. As silyl radicals there come into consideration, for example, radicals of formula —SiR₃, wherein the R radicals may have identical or different meanings and are unsubstituted or phenyl-substituted C₁-C₈alkyl, especially C₁-C₄alkyl, or unsubstituted or substituted phenyl and wherein the mentioned phenyl radicals may each be further substituted, for example by C₁-C₄alkyl, halo-substituted C₁-C₄alkyl, C₁-C₄alkoxy, nitro or by halogen. The alkylene radicals and silyl radicals mentioned for the protecting bridges may be substituted, for example, by one or two of the R radicals as defined above.

Especially preferred as protecting bridges are radicals of formulae 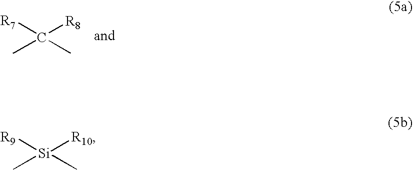
wherein R₇ and R₈ are each independently of the other hydrogen, unsubstituted or phenyl-substituted C₁-C₈alkyl or phenyl, and
R₉ and R₁₀ are each independently of the other unsubstituted or phenyl-substituted C₁-C₈-alkyl or phenyl,
   it being possible for each of the above-mentioned phenyl radicals to be further substituted, for example by C₁-C₄alkyl, halo-substituted C₁-C₄alkyl, C₁-C₄alkoxy, nitro or by halogen. The phenyl radicals are preferably unsubstituted.

R₇ and R₈ are preferably hydrogen, C₁-C₄alkyl, benzyl or phenyl, especially C₁-C₄alkyl, benzyl or phenyl. R₇ and R₈ are especially preferably methyl, tert-butyl or benzyl.

R₉ and R₁₀ are preferably C₁-C₄alkyl, benzyl or phenyl, especially C₁-C₄alkyl or benzyl. R₉ and R₁₀ are especially preferably methyl, tert-butyl or benzyl.

Preferred protecting bridges are those of formula (5a).

Y₁ and Y₂ are especially preferably each independently of the other hydrogen or together form a radical of formula (5a) or (5b), especially a radical of formula (5a). More especially Y₁ and Y₂ are hydrogen.

As organic radicals there come into consideration for X₁, for example, unsubstituted or substituted alkyl, alkenyl, alkynyl or phenyl radicals. Special mention may be made of unsubstituted or substituted C₁-C₁₂alkyl, C₃-C₁₂alkenyl, C₃-C₁₂alkynyl or phenyl radicals. In the case of X₁ preference is given to unsubstituted or substituted alkyl radicals, especially C₁-C₁₂alkyl radicals and preferably C₁-C₆alkyl radicals. An example of substituents of the alkyl radicals that may be mentioned is, for example, phenyl unsubstituted or further substituted in the phenyl ring by C₁-C₄alkyl, C₁-C₄alkoxy, nitro, halogen or by hydroxy. Examples of X₁ that may be mentioned include methyl, ethyl, n- or iso-propyl, n-, iso-, sec- or tert-butyl, allyl, benzyl, nitrobenzyl and hydroxybenzyl. X₁ is especially preferably C₁-C₄alkyl, especially butyl and preferably tert-butyl.

When the radical X₁ is a cation, the cation may be, for example, sodium or potassium, especially sodium.

X₁ is preferably hydrogen, unsubstituted or phenyl-substituted C₁-C₈alkyl or a cation. Especially preferably X₁ is a cation, such as sodium or potassium, especially sodium.

Z₁ is preferably bromine, chlorine, iodine, —OSO₂CF₃, —COCl, —B(OH)₂ or a mono- or di-ester derived from —B(OH)₂. Especially preferably Z₁ is bromine, chlorine or iodine, especially bromine, or —B(OH)₂ or a mono- or di-ester derived from —B(OH)₂. Bromine is of particular interest.

As mono-or di-ester derived from —B(OH)₂ there come into consideration for R₆ and Z₁ both cyclic and acyclic esters. Suitable mono- or di-ester derivatives of —B(OH)₂ are, for example, those of formula —B(OR′)₂, where the two R′ radicals may have identical or different meanings and are hydrogen, unsubstituted or phenyl-substituted C₁-C₈alkyl or unsubstituted or substituted phenyl, or wherein the two R′ radicals together form a C₁-C₈alkylene radical. Examples of substituents of the phenyl radical that may be mentioned include C₁-C₄alkyl, C₁-C₄alkoxy, amino, N-mono- or N,N-di-C₁-C₄alkyl, halogen, hydroxy and nitro. The R′ radicals are preferably hydrogen or C₁-C₄alkyl, preference being given to ethyl and especially methyl. It is also preferred that the two R′ radicals together form a C₁-C₈alkylene radical, especially a C₄-C₈alkylene radical. An example of such an alkylene radical that may be mentioned is the radical of formula —C(CH₃)₂—C(CH₃)₂—.

R₆ is preferably hydrogen, bromine, chlorine or iodine, especially hydrogen or iodine, preferably hydrogen.

As compound of formula (2) there is preferably used a compound of formula 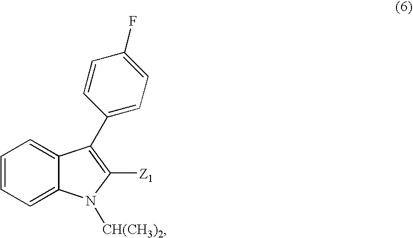
wherein the meanings and preferred definitions given above for Z₁ apply. Z₁ is especially bromine, —B(OH)₂ or a mono- or di-ester derived from —B(OH)₂, preferably bromine.

As compound of formula (3) there is preferably used a compound of formula 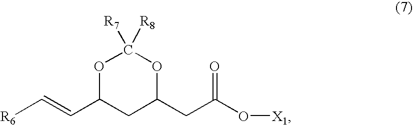
wherein the meanings and preferred definitions given above for R₆, R₇, R₈ and X₁ apply. R₆ is especially preferably hydrogen, bromine, chlorine or iodine, especially hydrogen. R₇ and R₈ are especially preferably each independently of the other hydrogen, unsubstituted or phenyl-substituted C₁-C₈alkyl or phenyl. It is more especially preferred to use the compound of formula (7) together with a compound of formula (6).

Compounds of formula (2) can be obtained, for example, by halogenating suitable compounds wherein Z₁ is hydrogen. The halogenation can be carried out according to generally customary methods. For bromination, mention may be made, for example, of Houben-Weyl, Methoden der organischen Chemie, volume 5/4, pages 233 ff, Georg Thieme Verlag, Stuttgart, 1960. Suitable for the bromination are, for example, elemental bromine, N-bromosuccinimide, pyridinium bromide perbromide or triphenylphosphine dibromide, in an inert, preferably halogenated, solvent, such as carbon tetrachloride, chloroform, chlorobenzene or dichlorobenzene. The bromination is generally carried out at a temperature of from −5 to 25° C., in the case of N-bromosuccinimide at about from 40 to 85° C. The starting compounds wherein Z₁ is hydrogen are known or can be obtained analogously to known processes, for example the processes indicated in U.S. Pat. No. 4,739,073. Compounds of formula (2) wherein Z₁ is —B(OH)₂ or a mono- or di-ester derived from —B(OH)₂ can be obtained analogously to known processes (e.g. starting from the compound of formula (2) wherein Z₁ is bromine).

Compounds of formula (3) are known (e.g. from U.S. Pat. No. 4,808,621) or can be obtained analogously to known processes.

As palladium catalyst there are preferably used olefinic palladium complex compounds.

Examples of such palladium catalysts that may be mentioned include compounds of formula 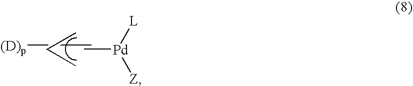
wherein L is a neutral ligand having electron donor properties, Z is an anionic ligand and D denotes substituents, and p is an integer from zero to five and defines the number of substituents on the allyl group;
and compounds of formula 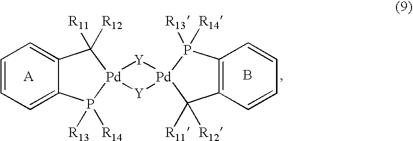
wherein
R₁₁, R₁₂, R₁₁′ and R₁₂′ are each independently of the others hydrogen, C₁-C₈alkyl, C₁-C₄-alkoxy, C₅-C₈cycloalkyl, C₁-C₄alkylcarbonyloxy, C₁-C₄alkoxycarbonyl, amino, N-mono- or N,N-di-C₁-C₄alkylamino, phenyl or halogen,
R₁₃, R₁₄, R₁₃′ and R₁₄′ are each independently of the others C₁-C₈alkyl, C₅-C₈cycloalkyl or unsubstituted or substituted phenyl, and
   the phenyl rings A and B are unsubstituted or substituted,
   and compounds of formula 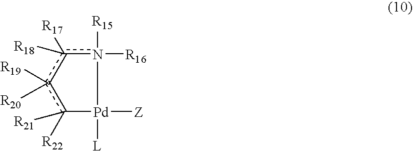
   wherein
(i) R₁₅ and R₁₆ together with R₁₇ and R₁₈ and R₁₉ and R₂₀, and together with the atoms to which they are bonded, form an unsubstituted or substituted quinolylene ring system, and R₂₁ and R₂₂ are each independently of the other hydrogen or an organic radical; or
(ii) R₁₇ and R₁₈ together with R₁₉ and R₂₀ and R₂₁ and R₂₂, and together with the atoms to which they are bonded, form an unsubstituted or substituted naphthylene ring system, and R₁₅ and R₁₆ are each independently of the other hydrogen or an organic radical; or
(iii) R₁₇ and R₁₈ together with R₁₉ and R₂₀, and together with the atoms to which they are bonded, form an unsubstituted or substituted phenylene ring, and R₁₅, R₁₆, R₂₁ and R₂₂ are each independently of the others hydrogen or an organic radical; or
(iv) R₁₉ and R₂₀, together with R₂₁ and R₂₂, and together with the atoms to which they are bonded, form an unsubstituted or substituted phenylene ring, and R₁₅, R₁₆, R₁₇ and R₁₈ are each independently of the others hydrogen or an organic radical; or
(v) R₁₅ and R₁₆, together with R₁₇ and R₁₈, and together with the atoms to which they are bonded, form an unsubstituted or substituted phenylene ring, and R₁₉ and R₂₀, together with R₂₁ and R₂₂, and together with the atoms to which they are bonded, form an unsubstituted or substituted phenylene ring; and
L and Z are as defined above;
   with the proviso that in cases in which R₁₅ and R₁₆ do not form an unsubstituted or substituted quinolylene or pyridylene ring system, R₁₅ and R₁₆, instead of being hydrogen or an organic radical, can also together form unsubstituted or substituted alkylene, which forms a ring together with the nitrogen atom.

L is a neutral ligand having electron donor properties. Suitable ligands are, for example, phosphine ligands of the tertiary phosphine type.

A suitable tertiary phosphine preferably contains from 3 to 40, especially from 3 to 18, carbon atoms. It preferably corresponds to the formula:
PR₂₃R₂₄R₂₅ (11)
wherein R₂₃, R₂₄ and R₂₅ are each independently of the others C₁-C₂₀alkyl, C₃-C₁₂cycloalkyl, C₂-C₁₁heterocycloalkyl, C₆-C₁₆aryl, C₇-C₁₆aralkyl or C₂-C₁₅heteroarylalkyl, it being possible for those radicals to be substituted by substituents selected from the group consisting of C₁-C₆alkyl, C₁-C₆alkoxy, C₁-C₆haloalkyl, C₆-C₁₆aryl, —NO₂, SO₃⁻, ammonium and halogen. The radicals R₂₃ and R₂₄ together can be unsubstituted or C₁-C₆alkyl-, C₁-C₆haloalkyl-, —NO₂— or C₁-C₆alkoxy-substituted tetra- or penta-methylene, which have been fused to one or two bivalent 1,2-phenylene radicals, R₂₅ being as defined above.

R₂₃, R₂₄ and R₂₅ as C₁-C₂₀alkyl are, for example, methyl, ethyl, n- or iso-propyl or n-, sec- or tert-butyl or straight-chain or branched pentyl, hexyl, heptyl, octyl, isooctyl, nonyl, tert-nonyl, decyl, undecyl or dodecyl.

R₂₃, R₂₄ and R₂₅ as C₃-C₁₂cycloalkyl are, for example, cyclopropyl, dimethylcyclopropyl, cyclobutyl, cyclopentyl or cyclohexyl.

R₂₃, R₂₄ and R₂₅ as C₂-C₁₁heterocycloalkyl preferably contain 4 or 5 carbon atoms and one or two hetero atoms from the group O, S and N. Examples include the substituents derived from oxirane, azirine, 1,2-oxathiolane, pyrazoline, pyrrolidine, piperidine, piperazine, morpholine, tetrahydrofuran and tetrahydrothiophene.

R₂₃, R₂₄ and R₂₅ as C₆-C₁₆aryl are, for example, mono-, bi- or tri-cyclic, e.g. phenyl, naphthyl, indenyl, azulenyl or anthryl.

R₂₃, R₂₄ and R₂₅ as C₂-C₁₅heteroarylalkyl are preferably such radicals that are, as heteroaryl, monocyclic or fused to a further heterocycle or to an aryl radical, e.g. phenyl, and preferably contain one or two, in the case of nitrogen up to four, hetero atoms from the group O, S and N. Examples of such heteroaryl radicals that may be mentioned include: furan, thiophene, pyrrole, pyridine, bipyridine, picolylimine, γ-pyran, γ-thiopyran, phenanthroline, pyrimidine, bipyrimidine, pyrazine, indole, coumarone, thionaphthene, carbazole, dibenzofuran, dibenzothiophene, pyrazole, imidazole, benzimidazole, oxazole, thiazole, dithiazole, isoxazole, isothiazole, quinoline, isoquinoline, acridine, chromene, phenazine, phenoxazine, phenothiazine, triazine, thianthrene, purine and tetrazole. C₂-C₁₅Heteroarylalkyl consists preferably of the mentioned heterocycles which substitute, for example, C₁-C₄alkyl radicals, depending on the length of the carbon chain where possible in the terminal position but alternatively in the adjacent position (1-position) or in the α-position (2-position).

R₂₃, R₂₄ and R₂₅ as C₇-C₁₆aralkyl preferably contain from 7 to 12 carbon atoms, e.g. benzyl, 1- or 2-phenethyl or cinnamyl.

Preference is also given to sterically demanding radicals R₂₃, R₂₄ and R₂₅, for example cyclic or branched, especially α,α-dibranched, and more especially α-branched, alkyl groups.

Special preference is given to those compounds (8) or (10) in which R₂₃, R₂₄ and R₂₅ are methyl, ethyl, n- or iso-propyl, n-, iso-, sec- or tert-butyl, 1-, 2- or 3-pentyl, 1-, 2-, 3- or 4-hexyl, cyclopentyl, cyclohexyl, phenyl, naphthyl or benzyl, e.g. (iso-C₃H₇)₃P, (C₅H₉)₃P, (C₆H₁₁)₃P and (C₆H₅)₃P.

As organic group there comes into consideration for the substituents of the compound of formula (10), for example, C₁-C₂₀alkyl, C₃-C₁₂cycloalkyl, C₆-C₁₆aryl or C₂-C₁₅heterocyclyl. As examples of such radicals, reference may be made to the corresponding radicals mentioned above for R₂₃, R₂₄ and R₂₅. Examples of substituents of such radicals that may be mentioned include: C₁-C₄alkyl, halo-substituted C₁-C₄alkyl, for example trifluoromethyl, C₆-C₁₆aryl, especially phenyl or naphthyl (C₆-C₁₆aryl, especially phenyl or naphthyl, being unsubstituted or substituted by halogen, carboxy, C₁-C₄alkoxycarbonyl, hydroxy, C₁-C₄alkoxy, phenyl-C₁-C₄alkoxy, C₁-C₄alkanoyloxy, C₁-C₄alkanoyl, amino, N—C₁-C₄alkylamino, N,N-di-C₁-C₄-alkylamino, N-phenyl-C₁-C₄alkylamino, N,N-bis(phenyl-C₁-C₄alkyl)amino, C₁-C₄alkanoyl-amino, halo-substituted C₁-C₄alkyl, for example trifluoromethyl, sulfo, cyano and nitro), hydroxy, C₁-C₄alkoxy, phenyl-C₁-C₄alkoxy, C₁-C₄alkanoyloxy, amino, N—C₁-C₄alkylamino, N,N-di-C₁-C₄alkylamino, N-phenyl-C₁-C₄alkylamino, N,N-bis(phenyl-C₁-C₄alkyl)amino, C₁-C₄alkanoylamino, carbamoyl-C₁-C₄alkoxy, N—C₁-C₄alkylcarbamoyl-C₁-C₄alkoxy or N,N-di-C₁-C₄alkylcarbamoyl-C₁-C₄alkoxy, amino, mono- or di-C₁-C₄alkylamino, halogen, for example fluorine, chlorine or bromine, carboxy, C₁-C₄alkoxycarbonyl, phenyl-, naphthyl- or fluorenyl-C₁-C₄alkoxycarbonyl, for example benzyloxycarbonyl, C₁-C₄alkanoyl, sulfo, C₁-C₄-alkanesulfonyl, for example methanesulfonyl (CH₃—S(O)₂—), phosphono (—P(═O)(OH)₂), hydroxy-C₁-C₄alkoxyphosphoryl or di-C₁-C₄alkoxyphosphoryl, carbamoyl, mono- or di-C₁-C₄alkylcarbamoyl, sulfamoyl, mono- or di-C₁-C₄alkylaminosulfonyl, nitro and cyano.

As C₁-C₂₀alkyl preference is given to C₁-C₈alkyl, especially C₁-C₄alkyl. As C₃-C₁₂cycloalkyl preference is given to unsubstituted or C₁-C₄alkyl-substituted cyclohexyl, especially unsubstituted cyclohexyl. As C₆-C₁₆aryl preference is given to phenyl or naphthyl, especially phenyl, it being possible for those radicals to be substituted as indicated above.

As unsubstituted or substituted quinolylene ring system in formula (10) there comes into consideration, for example, a quinolin-1,8-ene ring system, which may be substituted as indicated above for the organic radicals. Preference is given to the corresponding unsubstituted ring systems.

As unsubstituted or substituted pyridylene ring system in formula (10) there comes into consideration, for example, a pyridin-1,2-ylene ring system, which may be substituted as indicated above for the organic radicals. Preference is given to the corresponding unsubstituted ring systems.

As unsubstituted or substituted naphthylene ring system in formula (10) there comes into consideration, for example, a naphthyl-1,8-ene ring system, which may be substituted as indicated above for the organic radicals. Preference is given to the corresponding unsubstituted ring systems.

As unsubstituted or substituted phenylene in formula (10) there comes into consideration, for example, ortho-phenylene, which may be substituted as indicated above for the organic radicals. Preference is given to the corresponding unsubstituted phenylene.

In cases in which R₁₅ and R₁₆ do not form an unsubstituted or substituted quinolylene or pyridylene ring system and R₁₅ and R₁₆, instead of being hydrogen or an organic radical, can also together form unsubstituted or substituted alkylene, which forms a ring together with the nitrogen atom, the alkylene is preferably C₁-C₈alkylene, especially C₃-C₆alkylene and preferably pentamethylene (in which case a piperidine ring is formed).

An anionic ligand is, for example, the hydride ion (H⁻), or a ligand derived, for example, from inorganic or organic acids by removal of protons, e.g. halides (F⁻, Cl⁻, Br⁻ and I⁻ or anions of oxyacids or derivatives thereof, for example SnCl₃⁻, SnCl₅⁻, BF₄⁻, B(aryl)₄⁻, PF₆⁻, SbF₆⁻ or AsF₆⁻.

Anions of oxyacids are, for example, sulfate, phosphate, perchlorate, perbromate, periodate, antimonate, arsenate, nitrate, carbonate, the anion of a C₁-C₈carboxylic acid, for example formate, acetate, propionate, butyrate, benzoate, phenylacetate, mono-, di- or tri-chloro- or -fluoro-acetate, sulfonates, for example mesylate, ethanesulfonate, propanesulfonate, n-butanesulfonate, trifluoromethanesulfonate (triflate), unsubstituted or C₁-C₄alkyl-, C₁-C₄-alkoxy- or halo-substituted, especially fluoro-, chloro- or bromo-substituted, benzene-sulfonate or p-toluenesulfonate, e.g. benzenesulfonate, tosylate, p-methoxy- or p-ethoxy-benzenesulfonate, pentafluorobenzenesulfonate or 2,4,6-triisopropylbenzenesulfonate.

Especially preferred anionic ligands are H⁻, F⁻, Cl⁻, Br⁻, BF₄⁻, PF₆⁻, SnCl₃⁻, SbF₆⁻, AsF₆⁻, CF₃SO₃⁻, C₆H₅—SO₃⁻, 4-methyl-C₆H₅—SO₃⁻, 3,5-dimethyl-C₆H₅—SO₃⁻, 2,4,6-trimethyl-C₆H₅—SO₃⁻ and 4-CF₃—C₆H₅—SO₃⁻, acetate and cyclopentadienyl (Cp⁻). Special preference is given to acetate, Cl⁻, Br⁻ or I⁻. Acetate is more especially preferred.

Suitable substituents D remain unchanged under the conditions of the coupling reactions. The substituents may be selected as desired. Suitable substituents D are selected from the group of functional groups or derivatised functional groups consisting of amino, C₁-C₄alkyl-amino, C₁-C₄dialkylamino, hydroxy, oxo, thio, —NO₂, carboxy, carbamoyl, sulfo, sulfamoyl, ammonio, amidino, cyano, formylamino, formamido and halogen or are saturated or unsaturated, aliphatic, cycloaliphatic or heterocycloaliphatic radicals, carbocyclic or heterocyclic aryl radicals, fused carbocyclic, heterocyclic or carbocyclic-heterocyclic radicals, which may in turn be combined as desired with further of those radicals and substituted by the mentioned functional groups or derivatised functional groups.

The mentioned substituents and radicals may also be interrupted by one or more bivalent radicals from the group —O—, —S—, —C(═O)—O—, —O—C(═O)—, —C(═O)—N(C₁-C₄alkyl)-, —N(C₁-C₄alkyl)-C(═O)—, —S(═O)—, —S(═O)₂—, —S(═O)—O—, —S(═O)₂—O—, —O—S(═O)—, —O—S(═O)₂—, —S(═O)—N(C₁-C₄alkyl)-, —S(═O)₂—N(C₁-C₄alkyl)-, —(C₁-C₄alkyl)N—S(═O)—, —(C₁-C₄-alkyl)N—S(═O)₂—, —P(═O)—, —P(═O)—O—, —O—P(═O)— and —O—P(═O)—O—.

As aliphatic radicals there come into consideration for D, for example, the radicals mentioned above for R₁₅, R₁₆ and R₁₇ as C₁-C₂₀alkyl.

As cycloaliphatic radicals there come into consideration for D, for example, the radicals mentioned above for R₁₅, R₁₆ and R₁₇ as C₃-C₁₂cycloalkyl.

As heterocycloaliphatic radicals there come into consideration for D, for example, the radicals mentioned above for R₁₅, R₁₆ and R₁₇ as C₂-C₁₁heterocycloalkyl.

As carbocyclic or heterocyclic aryl radicals there come into consideration for D, for example, the radicals mentioned above for R₁₅, R₁₆ and R₁₇ as C₆-C₁₆aryl, C₇-C₁₆aralkyl and C₂-C₁₅-heteroarylalkyl.

The radicals D are especially preferably hydrogen, C₁-C₄alkyl, halogen or phenyl, which may be substituted as indicated above.

Preferably the index p has the values 0, 1 or 2, especially 0.

Suitable olefinic palladium complex compounds (8) having substituents on the allyl group are illustrated by the following structural formulae: 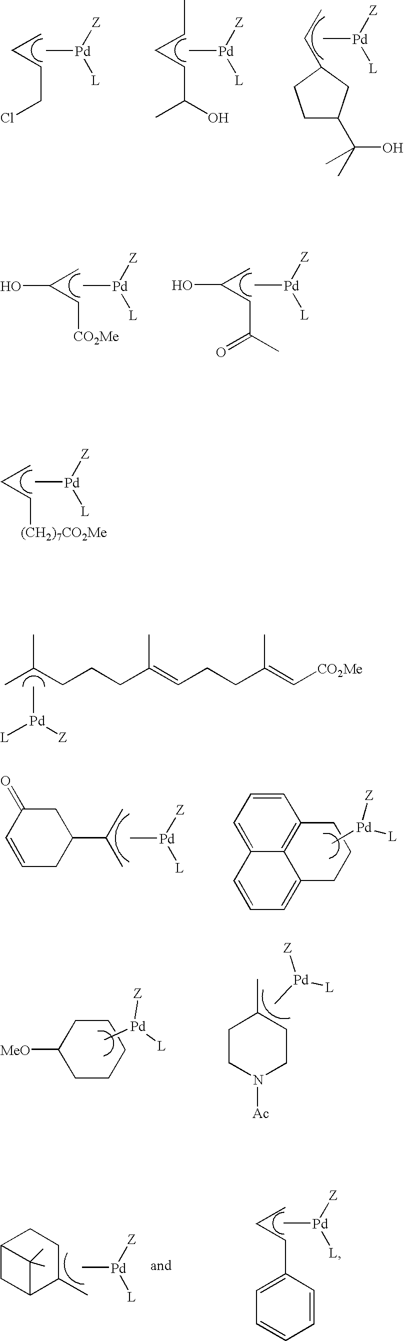
wherein Z and L are as defined and are preferably tricyclohexyiphosphine or triisopropylcyclophosphine and halogen, for example chlorine, bromine or iodine.

The substituents of the allyl group may, however, also be bonded with one another to form polynuclear bridged complexes according to the following structure:

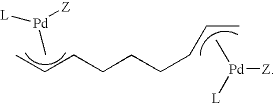

Preference is given to olefinic palladium complex compounds (8) without substituents on the allyl group, which is bonded to palladium (index p is zero), and wherein L is the tricyclohexyl-phosphine or triisopropyilcyclophosphine group and X is halogen, for example chlorine, bromine or iodine.

In addition to the compounds of formula (8) there also come into consideration those of formula 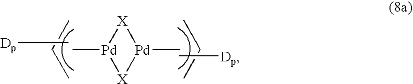
wherein the meanings and preferred definitions given above for D, X and p apply. The compounds of formula (8a) are added together with the ligand, the palladium complex being formed in situ.

Suitable palladium catalysts of formulae (8) and (8a) are known (e.g. from WO-A-99/47474) or can be obtained analogously to known palladium catalysts.

Examples of substituents of the phenyl rings A and B of the compounds of formula (9) that may be mentioned include C₁-C₄alkyl, C₁-C₄alkoxy, C₅-C₈cycloalkyl, C₁-C₄alkylcarbonyloxy, C₁-C₄alkoxycarbonyl, amino, N-mono- or N,N-di-C₁-C₄alkylamino, phenyl and halogen. As those substituents, preference is given to C₁-C₄alkyl, C₅-C₈cycloalkyl, such as cyclohexyl, or phenyl.

R₁₁, R₁₂, R₁₁′ and R₁₂′ are preferably each independently of the others hydrogen, C₁-C₄alkyl, C₅-C₈cycloalkyl, such as cyclohexyl, or phenyl.

R₁₃, R₁₄, R₁₃′ and R₁₄′ are preferably each independently of the others C₁-C₈alkyl, especially C₁-C₄alkyl, C₅-C₈cycloalkyl such as cyclohexyl, or unsubstituted or C₁-C₄alkyl-substituted phenyl.

For X there come into consideration the meanings and preferred definitions given above for the anionic ligand Z.

Palladium catalysts of formula (9) are known (e.g. from EP-A-0 690 046) or can be obtained analogously to known palladium catalysts.

Suitable palladium complex compounds of formula (10) are illustrated by the following structural formula: 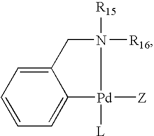
wherein the meanings and preferred definitions given above for R₁₅, R₁₆, Z and L apply. In those formulae preferably R₁₅ and R₁₆ are C₁-C₄alkyl, especially methyl, L is P(phenyl)₃ or P(isopropyl)₃ and Z is OAc.

In addition to the compounds of formula (10) there also come into consideration those of formula 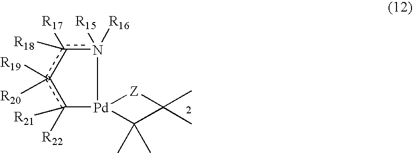
wherein for the substituents the meanings and preferred definitions given above apply.

The compounds of formula (12) are added together with the ligand, the palladium complex being formed in situ.

Special preference is given to suitable compounds of formula

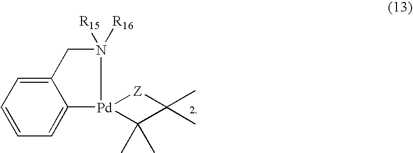

The compounds of formula (10) can be obtained analogously to known processes. For example, they may be obtained by the reaction of a compound of formula 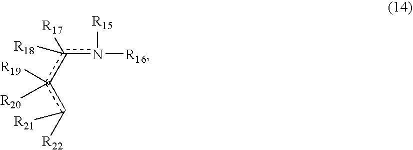
wherein the substituents are as defined above,
with a palladium salt of formula
Pd(Z)₂ (15),
wherein Z is as defined above, in a suitable solvent, especially a halogenated, preferably chlorinated, hydrocarbon, preference being given to C₁-C₄alkylhalides, such as chloroform or methylene chloride, at a temperature of, for example, from 0 to 50° C., especially from 20 to 30° C., and isolation of the resulting complex (generally, especially in the case when Z is C₁-C₄alkylcarbonyl, a dimeric compound of formula (12) bridged by way of Z is obtained). The resulting compound can then be reacted with a ligand
L (16),
wherein the meanings given above apply, optionally directly in situ in the reaction mixture used for the catalysis. The reaction is carried out in a suitable solvent, for example an ether, such as tetrahydrofuran, at a temperature of, for example, from 0 to 50° C., especially from 20 to 30° C. The resulting complex can then be used either directly or after isolation.

The starting materials for the preparation of the compound of formula (10) are known or can be obtained analogously to known processes.

As palladium catalysts special preference is given to those of formulae (8), (8a), (10) and (12), especially those of formulae (10) and (12). Those of formula (10) are of particular interest.

The reaction conditions for the coupling reactions of the compounds of formula (2) with those of formula (3) are described in the literature and correspond to the reaction conditions known for so-called Suzuki and Heck coupling reactions.

The process according to the invention can be carried out by using either the compound of formula (2) or the compound of formula (3) as initial charge, or by introducing both compounds.

The term “catalytic amount” preferably means an amount of about from 0.0001 to 15 mol %, especially from 0.01 to 10 mol % and more especially from 0.1 to 10 mol %, based on the amount of substrate used.

The molar ratio of the reaction partners in the coupling reactions of compounds of formula (2) to the compounds of formula (3) is generally in the range from 0.5:1 to 1:10, a ratio in the range from 0.5:1 to 1:5 being preferred. A ratio of from 1:1 to 1:2 is especially preferred. The reaction is carried out at a temperature ranging from with cooling up to the boiling temperature of the solvent, especially from room temperature up to the boiling temperature of the solvent (reflux conditions). Preference is given to temperatures of from 25 to 170° C., especially from 50 to 150° C. and preferably from 100 to 150° C. Suitable solvents are customary, especially relatively high-boiling, solvents, for example nonpolar aprotic solvents, e.g. xylene or toluene, or polar aprotic solvents, e.g. dimethylformamide. The obtainable reaction product can be worked up and isolated in a manner known per se. Mention may be made of customary purification methods, for example removal of the solvent and optionally subsequent separation processes, e.g. fine distillation, recrystallisation, preparative thin-layer chromatography, column chromatography, preparative gas chromatography etc.

Subsequent to the preparation of the compound of formula (4), the radicals Y₃ and Y₄ can be converted into the radicals Y₁ and Y₂ where Y₁ and Y₂ are hydrogen. That removal of the protecting groups can be carried out in conventional manner, for example by reaction under basic or acidic conditions. Removal of the protecting groups is preferably carried out subsequent to the preparation of the compound of formula (4).

When X₁ is hydrogen or an organic radical, X₁ can be converted into a cation, for example by hydrolysis.

The hydrolysis can be carried out, for example, by conventional basic hydrolysis of the esters. For that purpose, the compound of formula (4) is treated with about one mole of an inorganic base, for example an alkali metal hydroxide, e.g. potassium hydroxide or especially sodium hydroxide, in a mixture of water and a water-miscible organic solvent, for example a lower alcohol or an ether, such as methanol, ethanol or tetrahydrofuran, at a temperature of, for example, from 0 to 80° C. It is also possible to carry out the operation with slightly less than a stoichiometric amount of base and then to remove the excess ester by means of extraction with an organic solvent that is not miscible with water, e.g. tert-butyl methyl ether; freeze-drying can then be carried out. In order to form the free acid, the ester can also be hydrolysed in an acidic medium, it being possible for that hydrolysis to be carried out according to processes known per se. Hydrolysis is preferably carried out, preferably using sodium hydroxide, subsequent to the preparation of the compound of formula (4).

In dependence upon the optical purity of the compound of formula (3) used, the compounds of formula (1) can be obtained in the form of racemates or in the form of stereoisomerically pure compounds. Stereoisomerically pure compounds are to be understood here and hereinafter as compounds that are at least 60%, especially 80% and preferably 90%, pure. Such compounds are especially preferably at least 95%, preferably 97.5% and more especially 99% in stereoisomerically pure form.

For example, when corresponding stereoisomerically pure compounds of formula (3) are used, compounds of formula (1) can be obtained in pure form, especially in the (3R,5S) configuration given below:

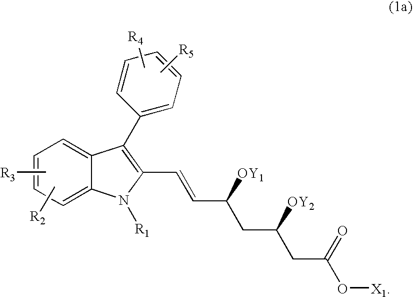

Further stereoisomers that may be mentioned include those of the corresponding (3R,5R), (3S,5S) and (3S,5R) configurations.

When a racemate is used as compound of formula (3), separation of the racemate can also be effected subsequent to the preparation of the compound of formula (1). The racemate can be separated into the optically pure antipodes, for example, by known processes for separating enantiomers, for example by means of preparative chromatography on chiral supports (HPLC) or by esterification and crystallisation with optically pure precipitants, e.g. with D-(−) or L-(−)-mandelic acid or (+)- or (−)-10-camphorsulfonic acid.

The present invention relates also to compounds of formula 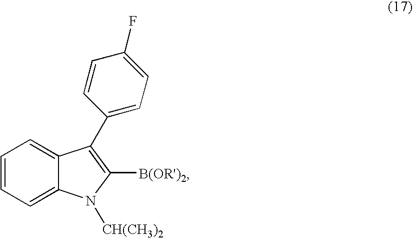
wherein for R′ the meanings and preferred definitions given above apply. The two R′ radicals preferably have identical or different meanings and are hydrogen, unsubstituted or phenyl-substituted C₁-C₈alkyl or unsubstituted or substituted phenyl, or the two R′ radicals together form a C₁-C₈alkylene radical.

As examples of substituents of the phenyl radical there may be mentioned C₁-C₄alkyl, C₁-C₄-alkoxy, amino, N-mono- or N,N-di-C₁-C₄alkyl, halogen, hydroxy and nitro. The R′ radicals are preferably hydrogen, benzyl or C₁-C₄alkyl, preference being given to ethyl or especially methyl. It is also preferred that the two R′ radicals together form a C₁-C₈alkylene radical, especially a C₄-C₈alkylene radical. As an example of such an alkylene radical there may be mentioned the radical of formula —C(CH₃)₂—C(CH₃)₂—.

The present invention relates also to compounds of formula 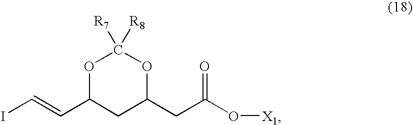
wherein for R₇, R₈ and X₁ the meanings and preferred definitions given above apply. R₇ and R₈ are especially each independently of the other hydrogen, unsubstituted or phenyl-substituted C₁-C₈alkyl or phenyl, especially C₁-C₄alkyl or benzyl, preferably C₁-C₄alkyl. X₁ is preferably C₁-C₄alkyl.

The following Examples illustrate the invention:

### EXAMPLE 1

### 2-Bromo-3-(4-fluoro-phenyl)-1-isopropyl-1H-indole

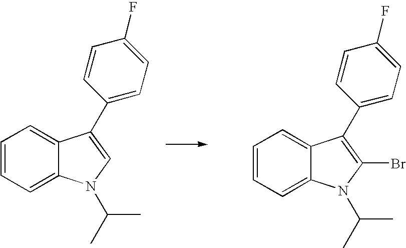

20 g (78.95 mmol) of 3-(4-fluoro-phenyl)-1-isopropyl-1H-indole, 200 ml of THF and 200 ml of chlorobenzene are introduced into a 1.5 liter sulfonating flask equipped with an anchor stirrer, thermometer and nitrogen supply, and the mixture is cooled to 3° C. with stirring. 26.58 g (78.95 mmol) of pyridinium bromide perbromide are then added, and stirring is carried out for 1.25 hours at 3° C. Thereafter, in the course of 10 minutes, 680 g of a 5% sodium hydrogen carbonate solution are added dropwise. The phases are separated and the aqueous phase is extracted three times with 150 ml of chlorobenzene. The combined organic phases are washed twice with 340 ml of 5% sodium hydrogen carbonate solution and twice with 220 ml of water, dried over magnesium sulfate, filtered and concentrated by evaporation. The brown residue is dissolved in 125 ml of methylene chloride; 125 ml of 94% ethanol are added, and the methylene chloride is distilled off at normal pressure. The solution is cooled slowly to room temperature, and then to 3° C., and the precipitate is filtered off, washed three times with 10 ml of ice-cold 94% ethanol and dried overnight at RT/125 T. Beige crystals are obtained having a melting point of from 110 to 111.5° C. Elemental analysis: found 4.95% H; 61.23% C; 4.04% N; 22.9% Br; 5.67% F. Theory 4.55% H; 61.46% C; 4.22% N; 24.05% Br; 5.72% F.

### EXAMPLE 2

### 1-Isopropyl-3-(4-fluorophenyl)-2-(4,4,5,5-tetramethyl-[1,3,2]dioxaborolan-2-yl)-1H-indole

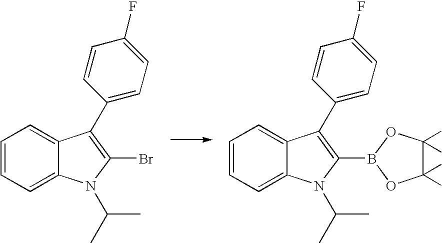

5.5 ml of a 1.6M solution of n-butyllithium in hexane are added, at a temperature of −78° C., to a solution of the above indole bromide (2.65 g) in 60 ml of a mixture of dry tetrahydro-furan/diethyl ether (ratio by volume 1:1). Stirring is carried out at a temperature of −78° C. for 15 minutes. A solution of 2-ethoxy-4,4,5,5-tetramethyl-1,3,2-dioxaborolane (2.4 ml) in diethyl ether (2 ml) is then added. The reaction mixture is heated to room temperature in the course of about 2.5 hours and then diluted with diethyl ether. The organic phase is washed with saturated sodium chloride solution, dried over Na₂SO₄ and is then concentrated by evaporation. The desired product is obtained in the form of yellowish crystals (3.0 g, 100%).

¹H-NMR (CDCl₃): 1.27 (s, 12H); 1.69 (d, J=7.0, 6H); 5.08-5.20 (m, 1H); 7.05-7.12 (m, 3H); 7.21-7.26 (m, 1H); 7.44-7.49 (m, 2H); 7.55-7.61 (m, 2H).

### EXAMPLE 3

### (4R,6S)-3-[6-(2-Iodo-vinyl)-2,2-dimethyl-[1,3]dioxan-4-yl]-acetic acid tert-butyl ester

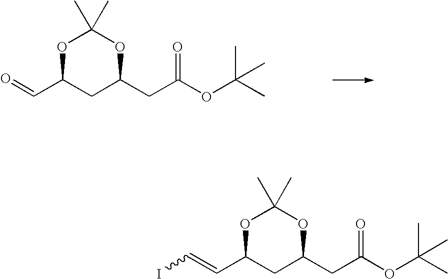

A solution of the above aldehyde (990 mg) and CHl₃ (2.26 g) in tetrahydrofuran (18 ml) is added at a temperature of 0° C., under argon, to a suspension of dry CrCl₂ (2.83 g) in dry tetrahydrofuran (36 ml). The reaction mixture is stirred at room temperature for 16 hours. The reaction is then stopped by the addition of water and extraction is carried out with diethyl ether. The organic phase is washed with saturated sodium chloride solution and dried over Na₂SO₄, and the solvent is removed under reduced pressure. The crude product is purified by chromatography (hexane/AcOethyl 1:1). The vinyl iodide (470 mg, 32%) is obtained in the form of a yellow oil (7:3 ratio of E/Z).

¹H-NMR (CDCl₃): 1.21-1.39 (m, 1H); 1.40 (s, ˜3H); 1.44 (s, 6.3H); 1.45 (s, ˜3H); 1.46 (s, 2.7H); 1.53 (s, 0.3H); 1.56-1.78 (m, 1H); 2.29 (dd, J=15.4, 6.3, 0.7H); 2.32 (dd, J=15.0, 6.2, 0.3H); 2.44 (dd, J=15.3, 7.1, 1H); 4.21-4.38 (m, ˜2H); 6.23 (dd, J=7.3, 7.3, 0.3 H, Z); 6.34 (dd, J=7.9. 0.9, 0.3H, Z); 6.39 (dd, J=14.7, 0.9, 0.7H, E); 6.52 (dd, J=14.7, 5.6, 0.7H, E).

### EXAMPLE 4

### [4R,6S]-{6-[2-(1-Isopropyl-3-(4-fluorophenyl)-1H-indol-2-yl)-vinyl]-2,2-dimethyl-[1,3]dioxan-4-yl}-acetic acid tert-butyl ester

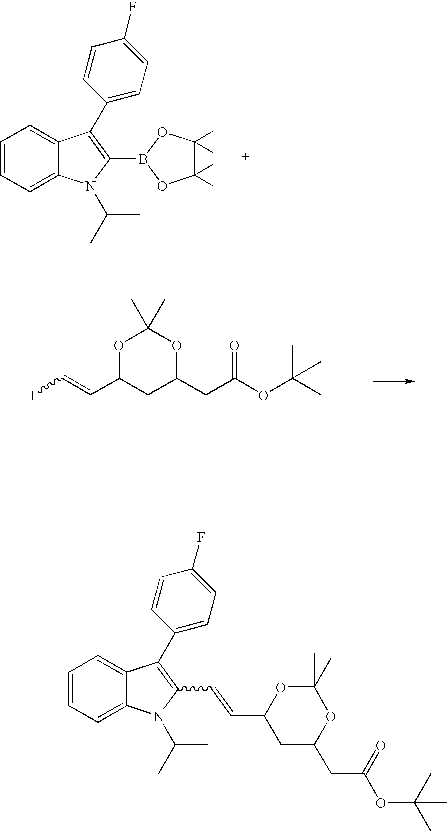

Water (6 ml), K₃PO₄ (427 mg) and Pd(dppf)Cl₂ (18 mg) [dppf=1,1′-bis(diphenylphosphino)-ferrocene] are added to a solution of the boronate (303 mg) and vinyl iodide (458 mg) in dimethoxyethane (6 ml). The reaction mixture is stirred under argon at a temperature of 60° C. for 40 hours. After cooling to room temperature, the reaction mixture is poured into water and extracted with AcOethyl. The combined organic phases are washed with saturated sodium chloride solution, dried over Na₂SO₄ and concentrated. The crude product is purified by chromatography (hexane/AcOethyl, 5:1). The desired product (235 mg, 46%) is obtained in the form of a yellowish foam (7:3 ratio of E/Z).

¹H-NMR (CDCl₃): 1.43 (s, 3H); 1.46 (s, 9H); 1.51 (s, 3H); 1.55-1.63 (m, 2H); 1.67 (d, J=7.0, 6H); 2.04 (dd, J=15.0, 5.0, 0.3H); 2.20 (dd, J=15.0, 7.8, 0.3H); 2.31 (dd, J=15.4, 6.2, 0.7H); 2.46 (dd, J=15.2, 7.0, 0.7H); 3.78-3.89 (m, 0.3H); 4.18-4.34 (m, 1H); 4.43-4.48 (m, 0.7H); 4.68-4.80 (m, 0.3H); 4.78-4.90 (m, 0.7H); 5.67 (dd, J=16.4, 5.9, 0.7H); 5.74 (dd, J=11.4, 8.2, 0.3H); 6.53 (d, J=11.1, 0.3H); 6.62 (d, J=16.1, 0.7H); 7.04-7.23 (m, 4H); 7.37-7.46 (m, 2H); 7.52-7.56 (m, 1.4H); 7.67 (d, J=8.0, 0.6H).

### EXAMPLE 5-16

In the following Examples, reference is made to the following compounds:
a) Starting materials 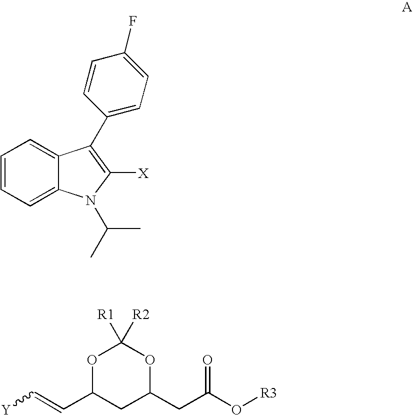
Compound A1: X=Br
Compound A2: X=B[OC(CH₃)₂C(CH₃)₂O]
Compound B1: R₁=R₂=CH₃; R₃=C(CH₃)₃; Y=H
Compound B2: R₁=R₂=CH₃; R₃=C(CH₃)₃; Y=I
b) Palladium catalysts 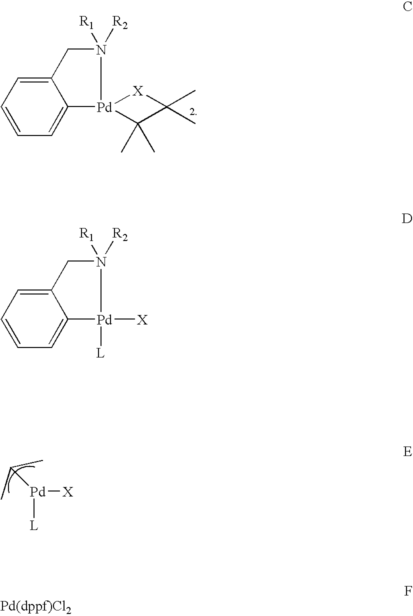
Compound C1: R₁=R₂=CH₃; X=OAc
Compound D1: R₁=R₂=CH₃; X=OAc; L=P(phenyl)₃
Compound E1: X=Br; L=P(isopropyl)₃
Compound F1: dppf=1,1′-bis(diphenylphosphino)ferrocene (commercially obtainable from Fluka)

General process procedure:

Compound A (1 molar equivalent) and compound B (1.2 molar equivalents, based on compound A) are dissolved in the respective solvent (10% solution) indicated in the following Tables 1(a) to 1(c). The base and the palladium catalyst are also added thereto. The reaction mixture is heated to the temperature shown in the Tables. After the reaction time indicated, the conversion and the yield are determined by means of HPLC. The results and reaction conditions are shown in the following Tables 1(a) to 1(c). The yield is determined by means of HPLC.

Generally used abbreviations:
Ac: acetyl
DMF: dimethylformamide
NMP: N-methylpyrrolidone

DME: dimethoxyethane

TABLE 1(a)

| Example | 5 | 6 | 7 | 8 |
|---|---|---|---|---|
| Compound A | A1 | A1 | A1 | A1 |
| Compound B | B1 | B1 | B1 | B1 |
| Palladium catalyst | D1 (1) | D1 (1) | D1 (1) | D1 (1) |
| (mol % Pd, based on | | | | |
| compound A | | | | |
| Base (molar | KOAc | NaOAc | K pivaloa- | K propio- |
| equivalent, based | (1.1) | (1.1) | te (1.1) | nate (1.1) |
| on compound A) | | | | |
| Solvent | DMF | DMF | DMF | DMF |
| Reaction temperature | 140° C. | 140° C. | 140° C. | 140° C. |
| Reaction time | 16 hours | 16 hours | 16 hours | 16 hours |
| Conversion, based on | 100% | 48% | 94% | 94% |
| compound A) | | | | |
| Yield | 68% | 33% | 62% | 62% |

TABLE 1(b)

| Example | 9 | 10 | 11 | 12 |
|---|---|---|---|---|
| Compound A | A1 | A1 | A1 | A1 |
| Compound B | B1 | B1 | B1 | B1 |
| Palladium catalyst | C1 (1) + | C1 (1) + | C1 (1) + | C1 (1) + |
| (mol % Pd, based on | P(n-butyl)₃(2) | P(cyclohexyl)₃ | P(isopropyl)₃ | P(phenyl)₃(2) |
| compound A) | | (2) | (2) | |
| Base (molar equivalent, | KOAc (1.1) | KOAc (1.1) | KOAc (1.1) | KOAc (1.1) |
| based on compound A) | | | | |
| Solvent | DMF | DMF | DMF | DMF |
| Reaction temperature | 140° C. | 140° C. | 140° C. | 140° C. |
| Reaction time | 3 hours | 3 hours | 3 hours | 3 hours |
| Conversion, based on | 60% | 62% | 55% | 84% |
| compound A) | | | | |
| Yield | 47% | 48% | 38% | 46% |
| Note: | catalyst is | catalyst is | catalyst is | catalyst is |
| | prepared in | prepared in | prepared in | prepared in |
| | situ | situ | situ | situ |

TABLE 1(c)

| Example | 13 | 14 | 15 | 16 |
|---|---|---|---|---|
| Compound A | A1 | A1 | A1 | A2 |
| Compound B | B1 | B1 | B1 | B2 |
| Palladium catalyst | D1 (1) | D1 (1) | E1 (1) | F1 (2.5) |
| (mol % Pd, based on | | | | |
| compound A) | | | | |
| Base (molar equivalent, | KOAc (1.1) | KOAc (1.1) | KOAc (1.1) | K₃PO₄ (2.5) |
| based on compound A) | | | | |
| Solvent | NMP | NMP | NMP | DME/H₂O in a |
| | | | | ratio by |
| | | | | volume of 1:1 |
| Reaction temperature | 140° C. | 200° C. | 200° C. | 60° C. |
| Reaction time | 18 hours | 1 hour | 1 hour | 40 hours |
| Conversion, based on | 94% | 96% | 96% | 98% |
| compound A) | | | | |
| Yield | 62% | 75% | 75% | 46% |

### EXAMPLE 17

### Erythro-(±)-E-7-[3-(4-fluoro-phenyl)-1-isopropyl-1H-indol-2-yl]-3,5-dihydroxy-hept-6-enoic acid tert-butyl ester

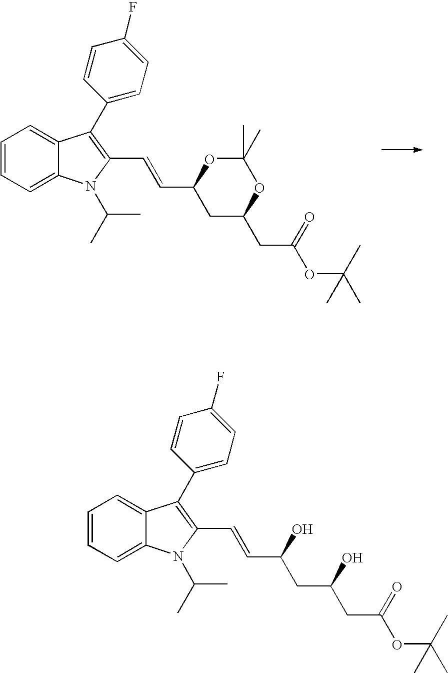

In a 5 ml round-bottomed flask, 0.1 g of erythro-(±)-E-(6-{2-[3-(4-fluoro-phenyl)-1-isopropyl-1H-indol-2-yl]-vinyl}-2,2-dimethyl-[1,3]dioxan-4-yl)-acetic acid tert-butyl ester and 8 mg of pyridinium p-toluenesulfonate are dissolved in 1.5 ml of acetonitrile; 0.1 ml of water is added and the clear solution is stirred at room temperature for 24 hours. The reaction mixture is then diluted with ethyl acetate, washed twice with saturated sodium chloride solution, dried over magnesium sulfate and concentrated by evaporation. 0.1 g of a beige solid is obtained, which, according to TLC, HPLC and NMR, corresponds to the product prepared as reference in the form of the tert-butyl ester analogously to U.S. Pat. No. 4,739,073, Example 5.

### EXAMPLE 18

### Erythro-(±)-(E)-7-[3-(4-fluoro-phenyl)-1-isopropyl-1H-indol-2-yl]-3,5-dihydroxy-hept-6-enoate sodium salt

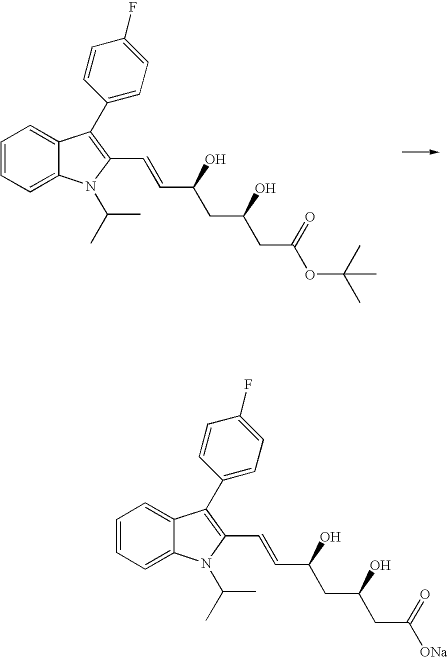

In a 10 ml three-necked round-bottomed flask equipped with a magnetic stirrer, thermometer, septum, syringe and nitrogen supply, 0.49 g of erythro-(±)-E-7-[3-(4-fluoro-phenyl)-1-isopropyl-1H-indol-2-yl]-3,5-dihydroxy-hept-6-enoic acid tert-butyl ester is hydrolysed according to O. Tempkin, Tetrahedron 31, 10659 (1997), there being obtained 0.35 g (77% of the theory) of a pale beige powder, the NMR of which corresponds to that of the commercial product.
Preparation Example for Palladium Catalyst:

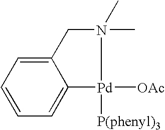

0.67 g of N,N-dimethylbenzylamine is slowly added to a solution of 1 g of Pd(OAc)₂ in 30 ml of chloroform. The reaction mixture is stirred for 2 hours and then filtered (silica). The resulting yellow solution is concentrated in vacuo and the resulting oil is suspended in a few ml of hexane. The yellow suspension is centrifuged and the resulting yellow powder is dried in vacuo. The compound of the symbolic formula 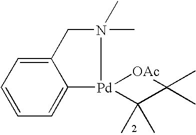
is obtained in quantitative yield. That dimer is dissolved in 10 ml of tetrahydrofuran, and 1 equivalent of triphenylphosphine is added. The reaction mixture is then stirred for 1 hour.

The resulting suspension is concentrated in vacuo and the white powder is washed with hexane. The desired product is obtained in a 90% yield in the form of a yellowish powder.

¹H NMR (δ in CDCl₃): 7.75 and 7.35 (2m, 15, PPh₃); 6.93 (d), 6.8 (t), 6.34 (m) (4, aromatic-H); 4.02 (d, 2.05 Hz, 2, CH₂N); 2.79 (d, 2.34 Hz, 6, NMe₂); 1.27 (s, 3, OAc).

³¹P NMR (δ in CDCl₃): 43

For preparation, see also Ryabov et al. in J. Chem. Soc., Perkin Trans. 1983, pages 1503-1508.

1. A process for the preparation of a compound of formula 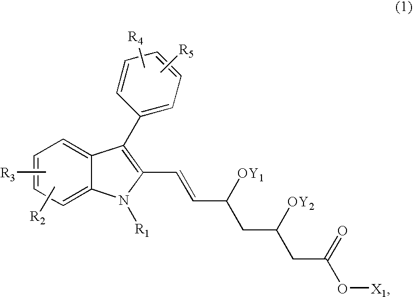
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈alkoxy, phenoxy or benzyloxy, or halogen,
Y₁ and Y₂ are each independently of the other hydrogen or a protecting group, or Y₁ and
Y₂ together form a protecting bridge, and
X₁ is hydrogen, an organic radical or a cation,
in which process a compound of formula 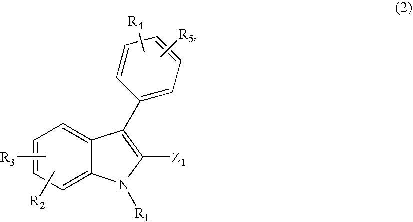
wherein R₁, R₂, R₃, R₄ and R₅ are as defined above, and
Z₁ is a leaving group,
is reacted, in the presence of a catalytically effective amount of a palladium catalyst, with a compound of formula 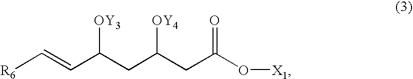
wherein R₆ is hydrogen, bromine, chlorine, iodine, —OSO₂CF₃, —COCl, —B(OH)₂ or a mono- or di-ester derived from —B(OH)₂,
Y₃ and Y₄ are each a protecting group, or Y₃ and Y₄ together form a protecting bridge,
and X₁ is as defined above,
to form a compound of formula 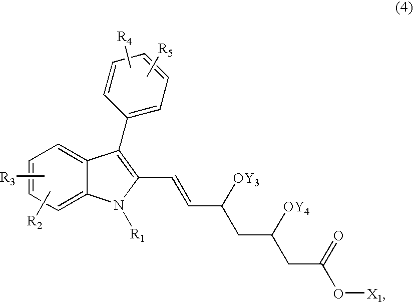
and optionally, the radicals Y₃ and Y₄ are converted into the radicals Y₁ and Y₂ where Y₁ and Y₂ are hydrogen.

2. A process according to claim 1, wherein R₁ is isopropyl.

3. A process according to claim 1, wherein R₂, R₃ and R₅ are hydrogen and R₄ is fluorine bonded in the 4-position.

4. A process according to claim 1, wherein Y₁ and Y₂ are each independently of the other hydrogen, C₁-C₄alkylcarbonyl or a silyl radical or Y₁ and Y₂ together form an unsubstituted or substituted alkylene or silyl radical.

5. A process according to claim 1, wherein Y₁ and Y₂ are each independently of the other hydrogen or together form a radical of formula 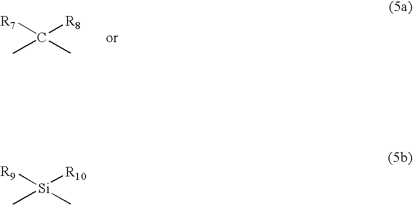
wherein
R₇ and R₈ are each independently of the other hydrogen, unsubstituted or phenyl-substituted C₁-C₈alkyl or phenyl, and
R₉ and R₁₀ are each independently of the other unsubstituted or phenyl-substituted C₁-C₈-alkyl or phenyl.

6. A process according to claim 1, wherein X₁ is hydrogen, unsubstituted or phenyl-substituted C₁-C₈alkyl or a cation.

7. A process according to claim 1, wherein X₁ is a cation.

8. A process according to claim 1, wherein R₆ is hydrogen, bromine, chlorine or iodine.

9. A process according to claim 1, wherein Z₁ is bromine, chlorine, iodine, —OSO₂CF₃, —COCl, —B(OH)₂ or a mono- or di-ester derived from —B(OH)₂.

10. A process according to claim 1, wherein as compound of formula (2) there is used a compound of formula 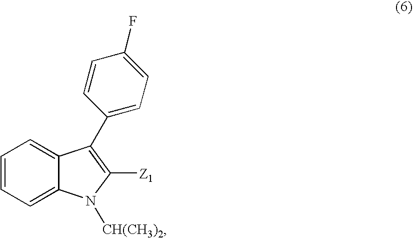
wherein Z₁ is bromine, —B(OH)₂ or a mono- or di-ester derived from —B(OH)₂, and as compound of formula (3) there is used a compound of formula 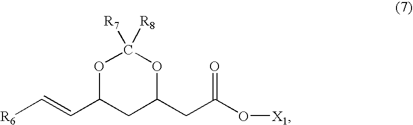
wherein R₆ is hydrogen, bromine, chlorine or iodine,
X₁ is as defined for claim 1, and
R₇ and R₈ are each independently of the other hydrogen, unsubstituted or phenyl-substituted C₁-C₈alkyl or phenyl.

11. A process according to claim 1, wherein there is used as palladium catalyst a compound of formula 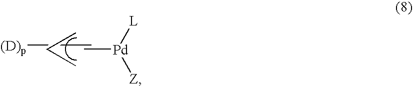
wherein L is a neutral ligand having electron donor properties, Z is an anionic ligand and D denotes substituents, and p is an integer from zero to five and defines the number of substituents on the allyl group;
or a compound of formula 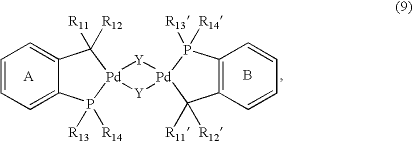
wherein
R₁₁, R₁₂, R₁₁′ and R₁₂′ are each independently of the others hydrogen, C₁-C₈alkyl, C₁-C₄-alkoxy, C₅-C₈cycloalkyl, C₁-C₄alkylcarbonyloxy, C₁-C₄alkoxycarbonyl, amino, N-mono- or N,N-di-C₁-C₄alkylamino, phenyl or halogen,
R₁₃, R₁₄, R₁₃′ and R₁₄′ are each independently of the others C₁-C₈alkyl, C₅-C₈cycloalkyl or unsubstituted or substituted phenyl, and
the phenyl rings A and B are unsubstituted or substituted,
or a compound of formula 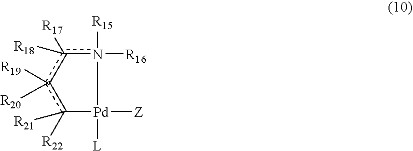
wherein
(i) R₁₅ and R₁₆ together with R₁₇ and R₁₈ and R₁₉ and R₂₀, and together with the atoms to which they are bonded, form an unsubstituted or substituted quinolylene ring system, and R₂₁ and R₂₂ are each independently of the other hydrogen or an organic radical; or
(ii) R₁₇ and R₁₈ together with R₁₉ and R₂₀ and R₂₁ and R₂₂, and together with the atoms to which they are bonded, form an unsubstituted or substituted naphthylene ring system, and R₁₅ and R₁₆ are each independently of the other hydrogen or an organic radical; or
(iii) R₁₇ and R₁₈ together with R₁₉ and R₂₀, and together with the atoms to which they are bonded, form an unsubstituted or substituted phenylene ring, and R₁₅, R₁₆, R₂₁ and R₂₂ are each independently of the others hydrogen or an organic radical; or
(iv) R₁₉ and R₂₀, together with R₂₁ and R₂₂, and together with the atoms to which they are bonded, form an unsubstituted or substituted phenylene ring, and R₁₅, R₁₆, R₁₇ and R₁₈ are each independently of the others hydrogen or an organic radical; or
(v) R₁₅ ad R₁₆, together with R₁₇ and R₁₈, and together with the atoms to which they are bonded, form an unsubstituted or substituted phenylene ring, and R₁₉ and R₂₀, together with R₂₁ and R₂₂, and together with the atoms to which they are bonded, form an unsubstituted or substituted phenylene ring; and
L and Z are as defined above;
with the proviso that in cases in which R₁₅ and R₁₆ do not form an unsubstituted or substituted quinolylene or pyridylene ring system, R₁₅ and R₁₆, instead of being hydrogen or an organic radical, can also together form unsubstituted or substituted alkylene, which forms a ring together with the nitrogen atom.

12. A process according to claim 11, wherein there is used as palladium catalyst a compound of formula (8) or (10).

13. A process according to claim 11, wherein there is used as palladium catalyst a compound of formula (10).

14. A process according to claim 1, wherein, subsequent to the preparation of the compound of formula (4), the radicals Y₃ and Y₄ are converted into the radicals Y₁ and Y₂ where Y₁ and Y₂ are hydrogen, and, when X₁ is hydrogen or an organic radical, X₁ is converted into a cation.

15. A compound of formula 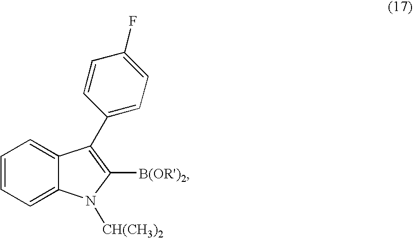
wherein the two R′ radicals have identical or different meanings and are hydrogen, unsubstituted or phenyl-substituted C₁-C₈alkyl or unsubstituted or substituted phenyl or wherein the two R′ radicals together form a C₁-C₈alkylene radical.

16. A compound according to claim 15, wherein the two R′ radicals have identical or different meanings and are hydrogen, benzyl or C₁-C₄alkyl, or the two R′ radicals together form a C₄-C₈alkylene radical.

17. A compound of formula 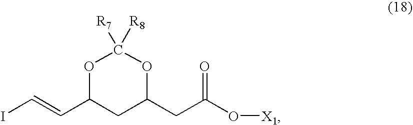
wherein R₇ and R₈ are each independently of the other hydrogen, unsubstituted or phenyl-substituted C₁-C₈alkyl, or phenyl, and
X₁ is unsubstituted or phenyl-substituted C₁-C₈alkyl.
